(19) United States
(12) Patent Application Publication
(10) Pub. No.: US 2004/0199258 A1
(43) Pub. Date: Oct. 7, 2004

(54) Modular acetabular cup and anchoring screw for fixing a prosphetic implant such as said acetabular cup

(76) Inventor: Frederique Macara, Carnon (FR)

Correspondence Address: HARRISON & EGBERT, 412 MAIN STREET, 7TH FLOOR, HOUSTON, TX, 77002, US

(21) Appl. No.: 10/484,548

(22) PCT Filed: Jul. 22, 2002
(86) PCT No.: PCT/FR02/02622

(30) Foreign Application Priority Data
Jul. 23, 2001 (FR) 01/09796

(51) Int. Cl. A61F 2/34

(52) U.S. Cl. 623/22.32; 623/22.38; 606/73

(57) ABSTRACT

The invention concerns a modular acetabular cup for surgical treatment for hip joint reconstruction in cases of total recovery of hip implant, and an anchoring screw for fixing a prosthetic implant such as said acetabular cup. The prosthetic acetabular cup comprises a support-plate (1) designed to be arranged in the acetabular cavity and anchored by means of an anchoring screw (4) including means (48) for adjusting the positioning and the locking in depth of said support-plate (1) and an adjustable space (2) locked thereon by a tapered shank assembly system (16, 21) and further a metal acetabular shell (3) designed to be assembled to said space (2) by a tapered shank assembly system (24, 33).

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

This invention relates to a modular prosthetic acetabulum for surgically treating the reconstruction of the hip joint in the cases of total restoring of hip prostheses, with or without bone-material deficiency, and in the cases of dysplasia, on the other hand, as well as an anchoring-screw device for fastening a prosthetic implant such as said acetabulum.

The aim of the acetabulum object of this invention is to restore all the bio-mechanical criteria of the joint by individually treating, thanks to its modularity, the basic elements which are the fastening and the orientation of the implant.

This principle allows meeting the needs for each situation that can arise and adapting to its requirements, thus authorizing fast loading of the patient.

### BACKGROUND OF THE INVENTION

The current technique for surgically treating the acetabulum reconstruction points out a deficiency at the level of both the intraossa anchoring of the existing prosthetic devices and their bio-mechanical positioning.

The anchoring with a limited primary fastening can indeed have, in the course of time, a harmful incidence on the integration of the implant, in view of the compressive and twisting forces which it is subjected to and which can give rise to a loosening and/or a breaking of the installed material.

The positioning of the implant, which is often dependent on, the state of the osseous acetabulum to be treated, does not allow a free orientation in all those planes that would allow restoring normal bio-mechanics of the joint, which can thus be prejudicial to the stability of the joint, very often causing dislocation and/or loosening of the implant.

### BRIEF SUMMARY OF THE INVENTION

The modular prosthetic acetabulum object of this invention allows coping with these deficiencies by bringing a global solution for the surgical treatment of the hip joint reconstruction by taking into consideration its mechanical, anatomical and biological requirements.

The modular prosthetic acetabulum object of this invention is mainly characterized in that it includes, on the one hand, a supporting plate aimed at being arranged in the acetabular cavity and at being anchored by means of anchoring screws having means allowing to ensure the adjustment of the positioning and the locking in depth of said supporting plate; on the other hand, a swiveling spacer locked on the latter by a Morse-tapers type assembling system; and still on the other hand, a metal cup aimed at being assembled to said spacer by means of a Morse-tapers type assembling system.

This prosthetic acetabulum allows to individually treat, on the one hand, the primary and secondary fastening, namely thanks to the characteristics of the anchoring screws, and, on the other hand, the anatomical orientation of the implant thanks to its broad modularity and, the stability of which is made reliable by the Morse-tapers type system for assembling each component the prosthetic acetabulum is comprised of.

The primary fastening ofthis prosthetic acetabulum is indeed ensured by the supporting plate, through anchoring screws which allow an anchoring in a healthy osseous area by using all the available supports, without any orientation constraint.

According to an additional feature of the prosthetic acetabulum according to the invention, the supporting plate includes at least an internal acetabular supporting leg provided with eyelets allowing an intra-acetabulum anchoring of said supporting plate by means of the anchoring screws.

According to another additional feature of the prosthetic acetabulum according to the invention, the supporting plate includes at least an external supra-acetabular leg provided with eyelets for receiving fastening screws of a spongy or cortical type, as well as an obturator hook capable of enduring a sub-acetabular fitting.

The multiplication of the anchoring and resting points of the support plate in the peripheral area thanks to the supra-acetabular legs and the obturator hook and in the internal area thanks to the acetabular legs allows achieving a maximal stability of said supporting plate and, hence, of the implant.

It should be noted that in the cases of an important bone destruction, the supra-acetabular fastening legs can also be fastened by means of anchoring screws identical to those being used for fastening the acetabular legs.

This association ofanchoring screws and supporting plate represents a real osteosynthesis and its primary mechanical stability remains an essential element of the life expectancy of the reconstruction, and authorizes fast loading of the patient.

According to another additional feature of the prosthetic acetabulum according to the invention, it includes a double layer, titanium and hydroxyspatite, osteoconductive coating.

The secondary fastening ofthis prosthetic acetabulum is optimized, on the one hand, through filling the acetabular cavity with grafts and/or bone substitutes, taking part in the reconstruction of the destroyed bone stock, and, on the other hand, thanks to the double osteoconductive coating which can be applied primarily on the back of the acetabular leg or legs of the supporting plate, as well as on the back of the metal cup which is accommodated in said supporting plate through the spacer.

According to another additional feature of the prosthetic acetabulum according to the invention, the spacer includes, on the one hand, a male cone located at its base and locking itself onto a female cone the supporting plate includes; and, on the other hand, a female cone located on its upper portion, which allows it to receive the male cone, the cup includes, which authorizes the orientation of the latter as needed, irrespective of the position of said supporting plate.

Thus, the spacer is designed swiveling in all planes, so as to act as an interface between the supporting plate and the cup, and to allow to center the anatomic vertical resting point inside said cup and to thus increase the congruency and the size of the bearing surface.

Independently from the fastening of the supporting plate in the osseous acetabulum, the adjustment of the anatomical orientation of the prosthetic acetabulum is ensured by the metal cup which is swiveling over 180° in the vertical, anteroposterior plane, and by the swiveling spacer in the vertical and horizontal planes, which interposes itselfbetween the supporting plate and the metal cup.

These two components allow the very fine adjustment of the anatomical axes of the joint.

The Morse-tapers type assembling of the various components the prosthetic acetabulum according to the invention is comprised of ensures the locking of the whole end makes its long-term stability reliable.

According to another additional feature of the prosthetic acetabulum according to the invention, the spacer is provided, on its external portion, in the center of its widest portion, and over more than half of its circumference, with a locking collar comprising, at several locations, eyelets allowing the passing through of screws aimed at being screwed into tapped holes provided for in a peripheral shoulder the supporting plate includes, said, collar having, at the level of each one of said eyelets, a concave profile capable, in co-operation with said screws the heads of which have a bearing surface with a hemispherical profile, of allowing an orientation of said screws according to several axes.

According to a variant embodiment, the cup is provided, in its external equatorial area, over more than half of its circumference, and above its male cone, with a collar capable of allowing an additional locking of the plate, the spacer and the cup; to this end, said collar comprises, at several locations, eyelets allowing the passing through of screws aimed at being screwed into tapped holes provided for in a peripheral shoulder the supporting plate includes, said collar having, at the level of each one of said eyelets, a concave profile capable, in co-operation with said screws the heads of which have a bearing surface with a hemispherical profile, allowing an orientation of said screws according to several axes.

According to a first particular embodiment of the prosthetic acetabulum according to the invention, the cup has a completely blind, mirror-type internal portion, which allows the free mobility of a polymeric insert it accommodates, which insert has a hemispherical cavity for receiving the spherical head of a prosthetic femoral element.

This architecture favors the magnitude of movement of the joint.

According to a second particular embodiment of the prosthetic acetabulum according to the invention, the cup has a completely blind internal portion having a female portion for receiving a ceramic insert having a male conical profile, in order to perform a Morse-tapers type assembling, said insert including a hemispherical cavity aimed at receiving the spherical head of a prosthetic femoral element.

This architecture, in which the insert is fixed with respect to the cup, meets specific prescriptions, for example, for young and/or very active patients.

These various characteristics of the prosthetic acetabulum object of the invention guarantee the restoring of normal biomechanics, token of life expectancy of the new prosthesis, and allow a faster rehabilitation and return to active life of the patient.

The present invention also relates to an anchoring screw for fastening a prosthetic implant such as the above-mentioned prosthetic acetabulum, it is mainly characterized in that it includes means capable of ensuring the fine adjustment of the height of the bearing surfaces of said implant, and the anchoring of the latter in the bone tissue while ensuring a balanced adjustment of the compressive forces.

According to an additional feature of the screw according to the invention, it includes a body comprising a cylindrically shaped proximal portion extended, by a distal portion ending in a sharp tip and having a spongy, self-tapping outer thread, said proximal portion being axially provided with a tapped channel aimed at receiving a threaded piston that is, in turn, provided with a head and a tapped well aimed at receiving a locking counter-screw.

Like a mechanical jack, the threaded piston allows, according to its depth of insertion into the tapped channel, to adjust the height of the bearing surfaces of the prosthetic implant, while the counter-screw ensures the tightening of said implant.

The advantages and features of the device according to the invention will become clear from the following description with reference to the attached drawing, which represents several non-restrictive embodiments of it.

### BRIEF DESCRIPTION OF THE SEVERAL VIEWS OF THE DRAWINGS

In the attached drawing:

FIG. 1 is a schematic exploded partial view of a prosthetic acetabulum according to the invention.

FIG. 2 is a schematic, median cross-sectional partial view of a variant embodiment of the same prosthetic acetabulum.

FIG. 3 is a prospective view of a variant embodiment of a portion of the acetabulum of FIG. 2.

FIG. 4 is a prospective view of a variant embodiment of another portion of the same acetabulum.

FIG. 5 is a prospective view of a variant embodiment of another portion of the same acetabulum.

FIG. 6 is a schematic, exploded, partly cross-sectional view according to a longitudinal plane, of an anchoring screw according to the invention.

### DETAILED DESCRIPTION OF THE INVENTION

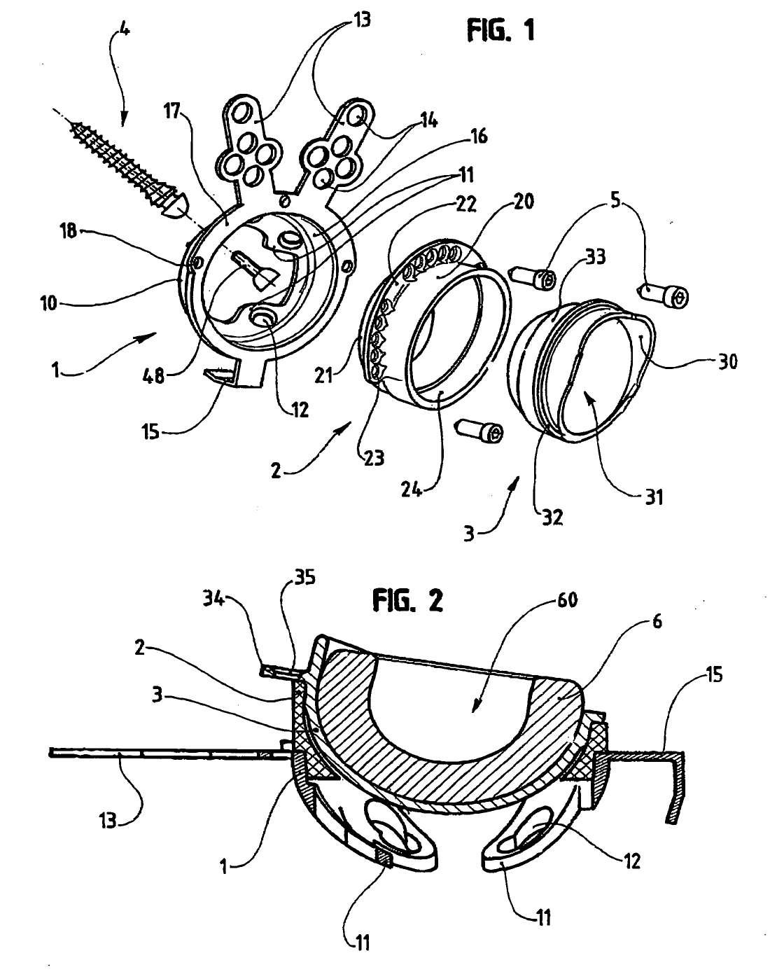

When referring to FIG. 1, one can see a prosthetic acetabulum according to the invention. It includes a supporting plate 1 aimed at being implanted in the acetabular cavity and at receiving a cup 3 mounted through a spacer 2.

The supporting plate 1 which is, preferably, made out of stainless steel or chromium-cobalt with or without a microporous double-layer coating, titanium and hydroxyapatite, forms the base of the prosthetic acetabulum.

It allows meeting the anatomical and mechanical requirements of the hip joint.

It is primarily in the form of a bearing ring 10 aimed at partially accommodating the spacer 2 and the cup 3, and which is provided with internal legs 11 with a lug 12 having a round shape and allowing the passing through of anchoring screws 4 which will be described hereafter. It should be noted that the internal legs 11 may include several eyelets 12, which can also have an elongated shape.

The supporting plate I also includes, in a known way, two external supra-acetabular legs 13 in which are provided eyelets 14 for the passing through of screws, not shown, for the fastening to the pelvic bone, as well as a hook 15 allowing an external support in the ischio-cotyloid notch.

The spacer 2 is in the form of a ring 20 that includes, externally, a distal area 21 with a conical profile, so as to allow a Morse-tapers type assembling with the supporting ring 10 which has, internally, a proximal area 16 with a complementary profile. It also includes, externally, in substantially median area, a peripheral collar 22 with a plurality of eyelets 23, and aimed at being placed adjacent to a collar 17 that includes the supporting plate 1 along its opening, and which is provided with tapped holes 18 capable of being put in front of the eyelets 23 and of receiving screws 5, with a view to, on the one hand, immobilizing in rotation the spacer 2 on the supporting plate I after having chosen an orientation and, on the other hand, locking this assembly.

The spacer 2 has a generally wedge-like shape, i.e. the planes of union with the support plate 1 and the cup 3, respectively, are convergent, so as to authorize, through the choice of its characteristics, an angular orientation allowing to center again the anatomical vertical resting point inside the cup 3 and thus to increase the congruency and the size of the bearing surface.

The cup 3 has a generally hemispherical shape with a slight cap-like projection 30; it includes a hemispherical cavity 31 aimed at receiving an insert 6, not shown in FIG. 1, but visible in FIG. 2, including, in turn, a hemispherical cavity 60 aimed at receiving the spherical head of a prosthetic femoral element.

The assembling of cup 3 is performed by means of a Morse-tapers type system, the spacer 2 thus includes a conical internal area 24, while the cup 3 includes a conical external area 33. Furthermore, the cup 3 includes a small equatorial rim 32 forming an unlocking element for the Morse taper, for its extraction.

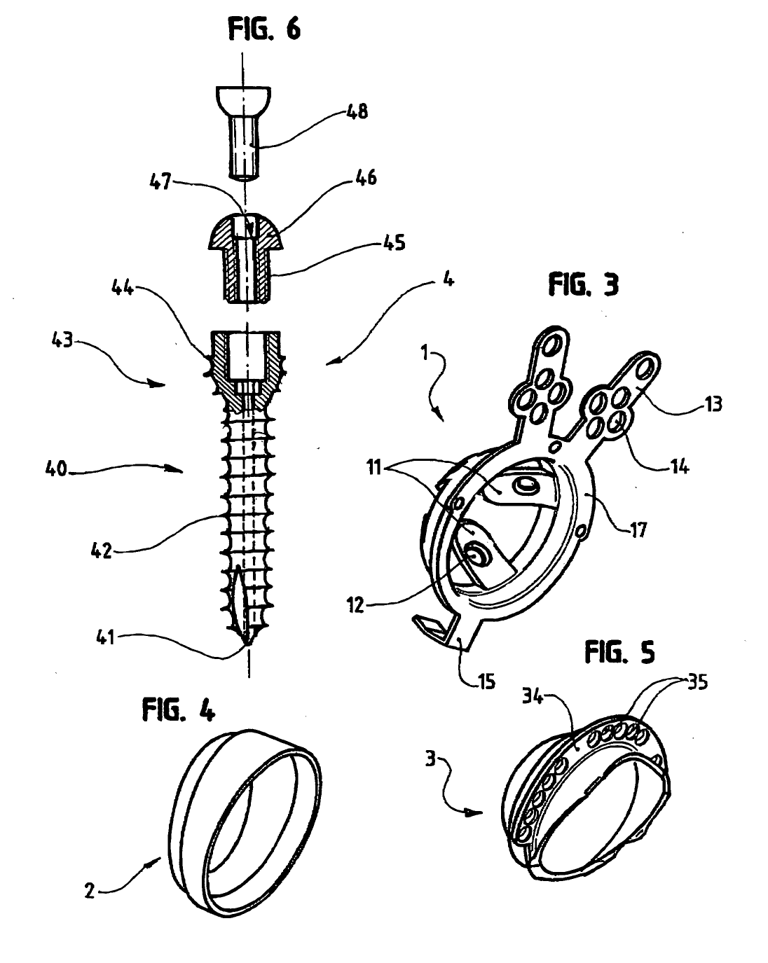

The prosthetic acetabulum according to the invention can have several variant embodiments; thus, when referring to FIG. 2 and to FIGS. 3, 4 and 5, one can see another prosthetic acetabulum according to the invention, also comprised of the assembling of a supporting plate 1, a spacer 2 and a cup 3, in which these various components each have a different construction.

In FIGS. 2 and 3, one can thus see that the supporting plate 1 has internal legs 11 having another, namely longer, shape.

In FIGS. 2 and 4 one can see a spacer 2 which does not include any collar.

And in FIGS. 2 and 5 one can see a cup 3 which includes externally, a collar 34 having a plurality of eyelets 35.

When referring now to FIG. 6, one can see an anchoring screw 4 which allows to perform the adjustment of the positioning of the supporting plate 1 in the bone cavit.-

This anchoring screw 4, which is preferably grooved, in order to facilitate its installation, comprises a threaded distal portion 40 having a tip 41 and a spongy outer thread 42, and a proximal portion 43 having the shape of a cylinder with, in the axial direction, a tapped channel 44 into which can be screwed a threaded piston 45 provided, in turn, with a head 46 and a tapped well 47 for receiving a locking counter-screw 43.

The threaded piston 45 allows, according to its depth of insertion into the tapped channel 44, to adjust the height of the bearing surfaces of the supporting plate 1, while the counter-screw 48 ensures the holding of said supporting plate 1.

It should be noted that the various threads have preferably reversed pitches.

After anchoring the distal portion into the bone material, screwing or unscrewing the piston 45 allows to adjust the height of the head 46 which is aimed at serving as a support for the supporting plate 1 and, more particularly, for an acetabular leg 11. After having determined the adequate length of insertion of the piston 45 into the channel 44, the supporting plate 1 is made integral by means of the locking counter-screw 48 screwed, into the well 47.

Of course, the proximal portion 43, the piston 47 and the counter-screw 48 are each provided with an operating die such an axial hollow die.

It should be noted that the screws 5, the locking counter-screw 48, as well as the fastening screws, not shown, of the external supra-acetabular legs 13 have heads the bearing surface of which has a curved, hemispherical profile, while the components 22, 34, 11 and 13, respectively, in which are provided the eyelets 23, 35, 12 and 14, respectively, have, at the level the letter, a countersinking with a complementary profile capable of authorizing the orientation of these screws, with respect to the components 22, 34, 11 and 13, according to the desired axis.

On the other hand, it should be noted that, advantageously, the base of the proximal portion 43, i.e. the area of connection with the distal portion 40 is truncated, and the outer thread 42 is extended in it, so as to ensure a safe anchoring.

The modular prosthetic acetabulum according to the invention is particularly intended for the surgical treatment of the reconstruction of the hip joint in the cases of total restoring of hip prostheses, in particular of great bone destruction, and, on the other hand, in the cases of dysplasia.

It should he noted that the use of the anchoring screw 4 is not limited to the fastening of a modular prosthetic acetabulum according to the invention, it can find another application in orthopedia.

1. Modular prosthetic acetabulum for surgical treatment of reconstruction of a hip joint in cases of total restoring of hip prostheses, comprising:
a supporting plate arranged in an acetabular cavity and anchored by means of anchoring screws with means to adjust positioning and locking in depth of said supporting plate (1);
a swiveling spacer locked on said supporting plate by a first Morse-tapers type assembling system; and,
a metal cup assembled to said spacer by a second Morse-tapers type assembling system (24, 33).

2. Modular prosthetic acetabulum according to claim 1, wherein said supporting plate further comprises an internal acetabular supporting leg with eyelets allowing an intra-acetabulum anchoring of said supporting plated by anchoring screws.

3. Modular prosthetic acetabulum according to claim 1, wherein said supporting plate further comprises an external supra-acetabular leg with eyelets receiving fastening screws of a spongy or cortical type, and an obturator hook ensuring a sub-acetabular fitting.

4. Modular prosthetic acetabulum according to claim 2, wherein said legs have, at the level of each of the eyelets, a concave profile capable, in co-operation with the screws inserted into said eyelets and heads of which have a bearing surface with a hemispherical profile, of allowing an orientation of said screws according to several axes.

5. Modular prosthetic acetabulum according to claim 1, further comprising a double layer, titanium and hydroxyapatite, osteoconductive coating.

6. Modular prosthetic acetabulum according to claim 1, wherein said spacer has a wedge-like shape, and is further comprised of a male cone located at a base thereof and lock onto a female cone and wherein said supporting plate further comprises a female cone located on an upper portion thereof, which allows receiving the male cone, and wherein said cup, which authorizes orientation of the latter as needed, irrespective of the position of said supporting plate

7. Modular prosthetic acetabulum according to claim 6, wherein said spaces is provided, on its external portion, in the center of its widest portion, and over more than half of its circumference, with a locking collar comprising, at several locations, eyelets allowing the passing through of screwse aimed at being screwed into tapped holes provided for in a peripheral shoulder the supporting plate includes, said collard having, at the level of each one of said eyelets, a concave profile capable, in co-operation with said screws the heads of which have a bearing surface with a hemispherical profile, of allowing an orientation of said screws according to several axes.

8. Modular prosthetic acetabulum according to claim 6, wherein said cup, is comprised of, in its external equatorial area, over more than half of its circumference, and above its male cone, a collar allowing an additional locking of the supporting platet, the spacer and the cup, to this end, said collar comprises, at several locations, eyelets allowing the passing through of screws aimed at being screwed into tapped holes provided for in a peripheral shoulder the supporting plate includes, said collar having, at the level of each one of said eyelets, a concave profile capable, in cooperation with said, screw the heads of which have a bearing surface with a hemispherical profile, allowing an orientation of said screws according to several axes.

9. Modular prosthetic acetabulum according to claim 1 wherein said cup has a completely blind, mirror-type internal portion, which allows the free mobility of a polymeric insert it accommodates, which insert has a hemispherical cavity for receiving the spherical head of a prosthetic femoral element.

10. Modular prosthetic acetabulum according to claim 1 wherein said cup has a completely blind internal portion having a female portion for receiving a ceramic insert having a male conical profile, in order to perform a Morse-tapers type assembling, said insert comprising a hemispherical cavity aimed at receiving the spherical head of a prosthetic femoral element.

11. Anchoring screw for fastening a prosthetic implant such as the prosthetic acetabulum according to claim 1, comprising: means ensuring fine adjustment of the height of the bearing surfaces of said implant, and the anchoring of the latter in the bone tissue while ensuring a balanced adjustment of the compressive forces.

12. Screw according to claim 11, further comprising: a body being comprised of a cylindrically shaped proximal portion extended by a distal portion ending in a sharp tip and having a spongy, self-tapping outer thread, said proximal portion being axially provided with a tapped channel aimed at receiving a threaded piston that is, in turn, provided with a head and a tapped well aimed at receiving a locking counter-screw.

13. Screw according to claim 12, wherein said counter-screw further comprises a head the bearing surface of which has a curved, hemispherical profile, aimed at co-operating with the component to be fastened which has, at the level of the eyelet aimed at receiving said counter-screw, a complementary profile, so as to authorize the orientation of said screw, with respect to said component, according to the desired axis.
